(19) United States
(12) Patent Application Publication
(10) Pub. No.: US 2010/0063418 A1
(43) Pub. Date: Mar. 11, 2010

(54) LANCING DEVICE FOR TAKING BLOOD, COMPRISING A LEG SPRING

(75) Inventor: Robert Wessel, Regensburg (DE)

Correspondence Address: BLACK LOWE & GRAHAM, PLLC, 701 FIFTH AVENUE, SUITE 4800, SEATTLE, WA, 98104, US

(73) Assignee: GERRESHEIMER WILDEN GMBH, Regensburg, DE (DE)

(21) Appl. No.: 12/529,902

(22) PCT Filed: Feb. 29, 2008
(86) PCT No.: PCT/EP2008/052486

§ 371 Date: Sep. 3, 2009

(30) Foreign Application Priority Data
Mar. 5, 2007 (DE) 10 2007 011 002.4
May 24, 2007 (DE) 10 2007 024 181.1

(51) Int. Cl. A61B 5/151

(52) U.S. Cl. 600/583

(57) ABSTRACT

A lancing device for taking blood for medical examinations, comprising a base body, at least one needle which is arranged therein and which can be extended outwards with a tip end, having a needle holding element which at least partially encompasses the needle, and a manual actuating element for releasing a movement of the needle together with the needle holding element relative to the base body, wherein the needle is spring-loaded and wherein at least one leg spring, the first end of which is attached to the base body, and by means of the second end of which the needle holding element can be moved along a predefined control curve section during a pressure release of the leg spring.

The invention relates to a lancing device for taking blood for medical examinations, comprising a base body (main body), at least one needle which is arranged therein and which can be extended outwards with a tip end, having a needle holding element which at least partially encompasses the needle, and a manual actuating element for releasing a movement of the needle together with the needle holding element relative to the base body (main body), wherein the needle is spring-loaded, according to the preamble of claim 1.

Lancing devices which are designed in a lancet-type manner and which are intended for lancing a skin surface for the purpose of subsequently taking blood are known in various forms. Often such lancing devices are designed for reuse and therefore are designed to be sterilised, and moreover clinical specialists are required in order to apply such lancing devices in a largely pain-free manner.

DE 196 17 000 C1 discloses a device comprising a needle which serves for taking blood, wherein a hollow cannula is designed with a sharpened cutting edge. Such devices are usually not intended for single use and, due to the needle shape, are not always pain-free during use. Moreover, they are designed preferably for clinical use.

A lancet for taking blood with a needle tip end protruding at the end region is likewise disclosed in DE 297 18 67 U1. The needle has an end region with the tip end which protrudes out from an axial end of a base body (main body) and which is embedded in a tubular attachment or connection region of a needle insertion head. The insertion head and the base body (main body) are formed in one piece. An axial pressure is exerted on this base body (main body) by means of a device (not shown) in order to allow the needle to penetrate to a certain depth into the body of the patient. The needle end region is then manually pulled out of the body of the patient. Such devices generally do not allow a pain-free lancing operation, since the handling by means of an additional pressure-exerting device allows only the insertion but not the removal of the needle.

Accordingly, the object of the present invention is to provide a lancing device for taking blood for medical examinations, which allows an almost pain-free lancing movement even by the actual person being examined, while being of simple and inexpensive design, the lancing device being intended for single use and being intended to allow a relatively large quantity of blood to be taken.

This object is achieved by the features of claim 1.

The core concept of the invention is that, in a lancing device for taking blood for medical examinations, comprising a base body (main body), at least one needle which is arranged therein and which can be extended outwards with a tip end, having a needle holding element which at least partially encompasses the needle, and a manual actuating element for releasing a movement of the needle together with the needle holding element relative to the base body (main body), use is made of a leg spring, the first end of which is attached to the base body (main body), and by means of the second end of which the needle holding element can be moved along a predefined control curve section (guiding curve section) during a pressure release of the leg spring. As a result of travelling along a control curve section, the situation is advantageously achieved whereby, in addition to an exclusively linear lancing movement which consists of a needle movement portion of being extended outwards and a needle movement portion of being retracted, at the same time a type of cutting movement can be obtained which additionally includes, by comparison with the linear movement portions, movement components which run perpendicular to the linear movement direction. As a result, a larger quantity of blood can be taken when the needle is inserted into the skin surface, since a cutting edge of the needle can be pivoted into the skin surface in the manner of blade during the cutting process which takes place and as a result the skin surface is not pierced at one point but rather is preferably gently cut so as to obtain a larger wound. This is particularly desirable when dealing with babies and small children, in which blood is taken from the heel. In this case, it is usually not possible to take enough blood by pricking with a needle having a round cross section, since the prick produces a wound that is too small.

By using a leg spring, a quick extension and retraction movement of the needle out of and into the base body (main body) is achieved in a simple and space-saving manner as soon as an actuating element is operated. In this case, it proves to be advantageous that the use of a leg spring avoids the transmission to the needle of any movement irregularities of the leg spring during a pressure release, since such a leg spring can be arranged so as to be able to slide at both ends relative to the base body (main body) and/or the needle holding element, so that during the pressure release it is possible for the ends to slide along the spring-loaded elements. Any undesired movement irregularities of the spring during the pressure release are thus prevented from being transmitted to the needle element which is intended to carry out the lancing movement. The use of the leg spring thus also helps to achieve a pain-free lancing operating by means of the lancing device according to the invention. Alternatively, the leg spring may be designed in such a way that the spring ends thereof are fixedly connected to the base body (main body) and/or to the needle holding element for example by means of chamfered spring ends or by means of spring eyes or other such elements. In this case, the wound region of the spring may be arranged in a freely movable, i.e. displaceable, manner within the base body (main body) or the housing, thereby resulting once again in no additional movement irregularities.

Such a leg spring may moreover already be in the pretensioned state when the lancing device is actuated for the first time. Due to the characteristic of leg springs, even after a long period of non-use, such a pretensioning of the leg spring does not have a disadvantageous effect on the spring force to be exerted, which is responsible for the lancing operation.

In addition, the use of such a leg spring allows a lancing aid to be constructed for single use since, by means of a suitable arrangement of the control curve or control curve section in combination with the leg spring ends, the entire lancing operation including the extension and retraction movement portion and the movement carried out during the cutting operation can be carried out by a pressure release movement of the leg spring ends.

The control curve section is preferably arranged as an arc-shaped or curved slot in a pivoting element which is mounted so as to pivot relative to the base body (main body), wherein the pivoting element is connected to the second end of the leg spring. Alternatively, the control curve section may be arranged as an arc-shaped or curved slot in the needle holding element, in which a cam arranged on the pivoting element engages. Instead of using a slot for the control curve section, the latter may be formed as a web in which a groove of complementary shape engages, said groove being used instead of the cam. The web accordingly travels along the groove.

The control curve section may likewise be arranged as an arc-shaped or curved groove on an inner wall of the base body (main body), wherein a cam of the needle holding element engages in the groove. Alternatively, the arc-shaped or curved groove may be arranged on the needle holding element and the cam may be arranged on the inner wall. When the control curve section is arranged on the inner wall of the base body (main body), this may likewise be designed as a web, over which there travels a groove arranged on the needle holding element.

When the control curve section is arranged in the pivotably mounted pivoting element, a guide cam of the needle holding element is preferably arranged so as to engage in the slot of the pivoting element, so that a pivoting movement of the pivoting element which is brought about as a result of the pressure release of the leg spring causes a linear extension and retraction movement of the needle holding element, optionally coupled to a cutting movement. This is achieved as a result of the fact that the slot is arranged in an arc-shaped manner within the pivoting element in such a way that a sliding of the arc-shaped slot along the cam of the needle holding element leads to a displacement movement of the needle holding element due to the arc shape of the slot.

When the control curve section is arranged as an arc-shaped or curved groove on an inner wall of the base body (main body), the needle holding element has a pivoting arm attached to the side, the first end of which is connected in a pivotable manner to the needle holding element and the second end of which is connected in a pivotable manner to the base body (main body), wherein the pivoting arm carries out a pivoting movement during a forward and backward displacement movement of the needle holding element so as thus to obtain a lateral cutting movement of the needle holding element and thus of the needle during the displacement of the needle holding element, i.e. a cutting movement oriented transversely to the extension and retraction direction of the needle holding element. Alternatively, the groove may be arranged on the needle holding element and the cam may be arranged on the inner wall.

Such a pivoting arm may be replaced by a groove arranged on the inner wall of the base body (main body) in the tip end region of the needle and thus of the needle holding element, in order thus to obtain a guidance by means of cams both in the front and in the rear region of the needle holding element with a desired movement in the extension and retraction direction and coupled to a pivoting movement.

In the lancing device with the curved groove arranged on the inner wall of the base body (main body), the manual actuating element is arranged at the upper end of the needle holding element located opposite the tip end and can be arrested in the base body (main body) designed as a housing. This takes place as a result of the needle holding element end, which represents the actuating element, being pushed into the base body (main body), whereupon the leg spring is released and triggers a pressure release. The needle holding element is then moved on the curved path of the groove-like control curve section, resulting in the lancing movement of the needle and of the needle holding element.

After the pressure release has been triggered, the actuated manual actuating element is arranged within the base body (main body) due to a lateral displacement relative to an opening, through which the manual actuating element previously protruded out of the base body (main body).

The arc-shaped or control curve section is preferably oriented in such a way that its ends are oriented towards the upper side of the base body (main body) in a manner pointing away from the tip end of the needle.

A rotation axle of the leg spring is arranged so as to run perpendicular to the longitudinal axis of the base body (main body) in the region of the lower side of the base body (main body), the retractable or extendable tip end of the needle being located at the lower side of the base body (main body).

Alternatively, a rotation axle of the leg spring may be arranged so as to run perpendicular to the longitudinal axis of the base body (main body) in the region of the upper side of the base body (main body).

Both possible arrangements of the leg spring allow a space-saving and thus compressed design of the single-use lancing device since, due to the construction of the leg spring, the arrangement thereof in combination with the displaceable needle holding element, which is preferably bar-shaped, allows a space-saving design.

The manual actuating element may alternatively lead to a locking of the pretensioned leg spring by means of a push-button arranged on the left-hand side and/or on the right-hand side of the base body (main body) which represents the housing, for which purpose the two housing halves merely have to be pressed together. This allows a sterile configuration of the single-use lancing aid, since it has an almost closed housing. The push-button is accordingly designed to be movable relative to one of the housing halves, in which a connection between the push-button and the housing half is formed by a very thin wall which changes its alignment when pressure is applied, in a manner similar to a “clicker”. The spacing of the other housing surfaces is not changed by this, and there is no risk of jamming of the components or elements arranged inside.

Further advantageous embodiments will emerge from the dependent claims.

Advantages and expedient features can be found in the following description in conjunction with the drawing, in which:

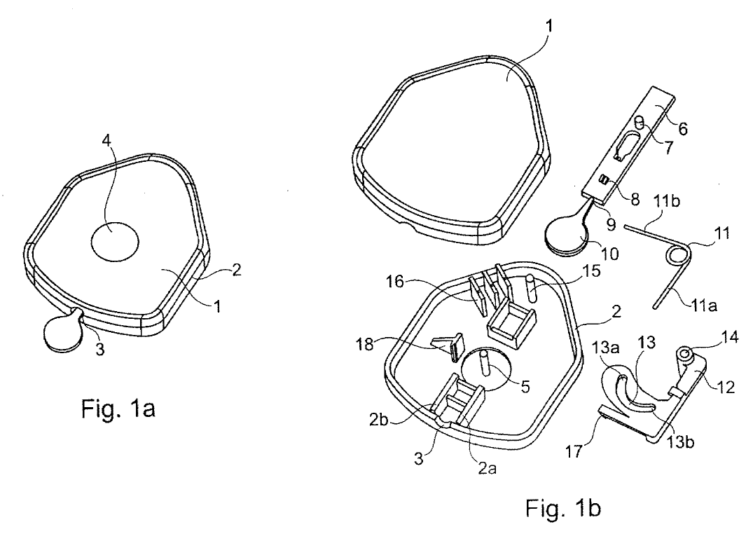

FIG. 1a shows a perspective view of a lancing device according to a first embodiment of the invention in the assembled state;

FIG. 1b shows a perspective view of a lancing device according to the first embodiment of the invention in the disassembled state;

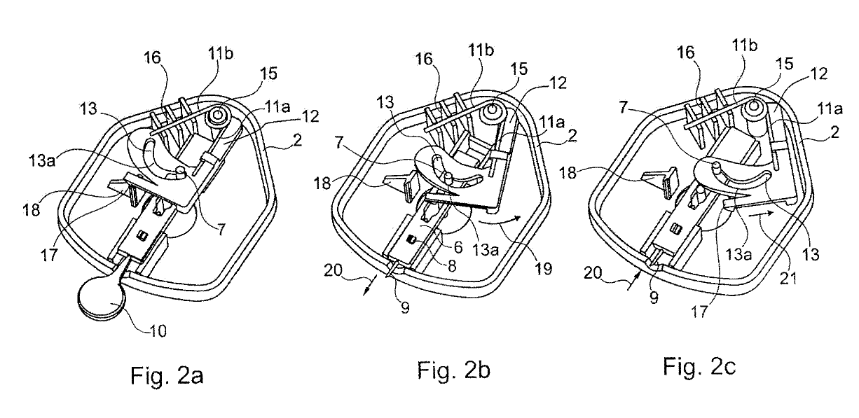

FIGS. 2a-c show perspective views of the lancing device according to the first embodiment of the invention before, during and after a lancing operation;

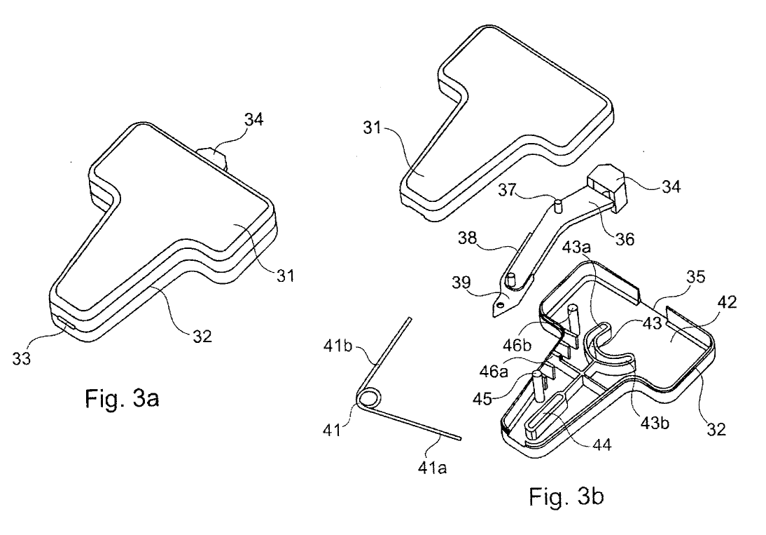

FIG. 3a shows a perspective view of the lancing device according to a second embodiment of the invention in the assembled state;

FIG. 3b shows a perspective view of the lancing device according to the second embodiment of the invention in the disassembled state;

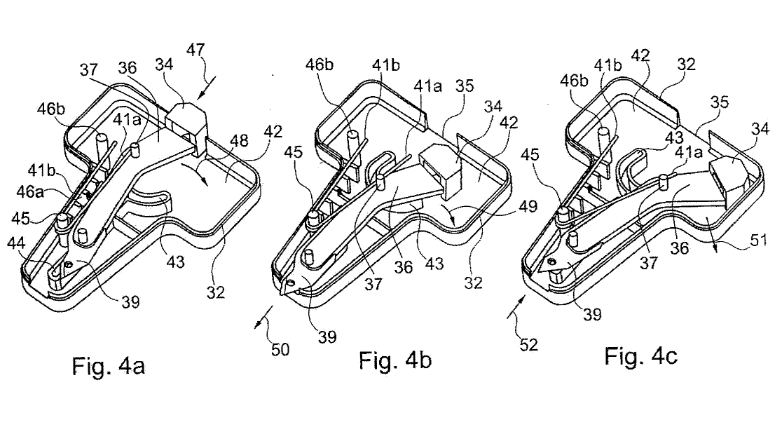

FIGS. 4a-c show a perspective view of the lancing device according to the second embodiment of the invention before, during and after the lancing operation;

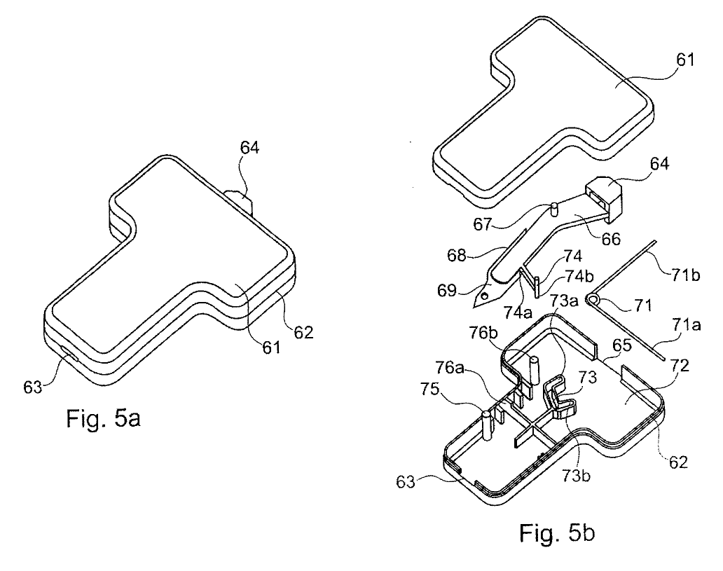

FIG. 5a shows a perspective view of the lancing device according to a third embodiment of the invention in the assembled state;

FIG. 5b shows a perspective view of the lancing device according to the third embodiment of the invention in the disassembled state; and

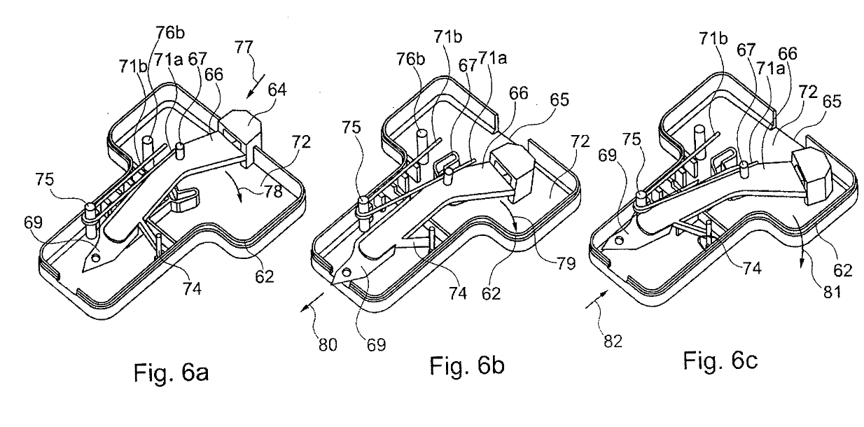

FIGS. 6a-c show a perspective, open view of the lancing device according to the third embodiment of the invention before, during and after a lancing operation.

FIGS. 1a and 1b show a perspective view of a lancing device according to a first embodiment of the invention in the assembled and disassembled state. A base body (main body) 1, 2 composed of two halves has an opening 3 arranged on the underside, through which a needle holding element can be extended and retracted.

A connecting bolt 5, which is arranged on the bottom half, serves to release a latching element. As the manual actuating element, a push-button 4 is formed by a very thin wall within the housing half, the alignment of which can be varied by applying pressure to the outside. The push-button may be arranged exclusively on one housing half or on both housing halves.

A needle holding element 6, which is preferably flat and bar-shaped, has on its surface a cam 7 for the engagement of an arc-shaped slot 13 of a pivoting element 12.

A needle 8 having a tip end 9 is provided at its front end with a protective cap 10 which is designed in a sterilised manner and can act on the needle while the lancing device is not in use. The protective cap 10 can be designed as sterile protection and can be removed by a rotary hand movement prior to actuation of the lancing aid. It may be made from a plastic material, such as an elastomer for example.

A leg spring 11 has a first end 11b and a second end 11a, wherein the first end is connected to the base body (main body) 2 via webs 16 which are arranged on the base body (main body) 16.

The second end 11a is connected to the pivoting element 12, which can be pivoted by means of a bore 14 arranged at the top about a rotation axle 15 arranged in a stationary manner on the housing 2.

The arc-shaped slot 13 has two ends 13a and 13b which point towards an upper end of the base body (main body) 1, 2.

Also arranged on the pivoting element is the latching hook 17 which cooperates with a hook 18 of complementary shape arranged on the base body (main body) 2 and can be released by means of the manual actuating element 4.

The needle holding element 6 runs within guide grooves 2b which are provided on the base body (main body) 2 and are arranged on an inner wall 2a of the base body (main body).

FIGS. 2a-c show a perspective, open view of the lancing device according to a first embodiment of the invention before, during and after the lancing operation. Before the lancing operation, as shown in FIG. 2a, the leg spring 11 is in the pretensioned state so that the pivoting element 12 is pivoted to the left and thus the cam 7 of the needle holding element 6 is arranged at the right-hand end of the arc-shaped slot 13.

As soon as the manual actuating element (not shown in any detail here) causes the leg spring 13 to be released, a pivoting movement of the pivoting element 12 takes place from left to right, as illustrated by an arrow 19. As a result, the cam 7 of the needle holding element 6 travels along the arc-shaped slot 13 and during the travel is arranged at a bottom point 13a of the slot-shaped slot, as shown in FIG. 2b. This leads to an excursion of the needle 8 by means of a downward displacement movement, as shown by the arrow 20.

In this way, the tip end 9 of the needle 8 is briefly extended out of the opening 3 of the base body (main body) 1, 2.

FIG. 2c shows the position of the needle and of the needle holding element after use of the lancing device, i.e. after the lancing operation has taken place. It can clearly be seen from this diagram that a pivoting movement of the pivoting element 12 from left to right, as illustrated by the arrow 21, has led to the situation whereby the cam 7 has arrived at the left-hand end of the arc-shaped slot 13.

The spring 11 is now in a pressure-released state, in which the first end 11b of the spring 11 is still arranged relative to the base body (main body) 2 in the webs 13 and the second end 11a of the spring 11 has pushed the pivoting element 12 outwards to the right.

During the transition of the movements of the displaced needle 8 from the extended position, as shown in FIG. 2b, into a position obtained after use of the lancing device, the needle tip 9 is retracted into the base body (main body) 1, 2, as shown by the arrow 20.

In FIGS. 3a and 3b, a lancing device according to a second embodiment of the invention is shown in a perspective view in the assembled and disassembled state. A base body (main body) 31, 31 comprises an upper half and a lower half and is provided at its exit side with an opening 33 for the extension and retraction of a needle tip. At the end of the base body (main body) 31, 32 located opposite the opening 33, a manual actuating element 34 is provided which is fixedly connected to a needle holding element 36.

The needle holding element 36 is arranged with the manual actuating element 34 at the top in an opening 35 of the base body (main body) 31, 32 and has at its lower side a needle 38 having a needle tip end 39 which is designed in the manner of a blade.

A first end of a leg spring 41 is arranged on webs 46a fixedly connected to the base body (main body) 2, and a second end 41a thereof is spring-loaded relative to the needle holding element 36.

The leg spring 41 is arranged in a rotatable manner on a bolt 45 which is fixedly connected to the base body (main body) 21 and serves as a rotation axle.

A bolt 46b which is likewise fixedly connected to the base body (main body) 32 serves as a stop for the first end 41b of the leg spring 41.

An inner wall 42 of the base body (main body) 32 has an arc-shaped groove 43 with a left-hand end 43a and a right-hand end 43b, wherein the two ends 43a, 43b point towards the opening 35, i.e. towards the top.

A further longitudinal groove 44 is arranged in the region of the opening 33 of the base body (main body), so that a guidance of the needle holding element 36 by means of cams 37 in these grooves is possible.

FIGS. 4a-4c show a perspective, open view of the lancing device according to the second embodiment of the invention before, during and after use or the lancing operation. In FIG. 4a, the needle holding element 36 is in a starting position in which the cam 37 is arranged at the left-hand first end 43a of the arc-shaped groove 43 relative to the base body (main body) 32 on the bottom side and the base body (main body) 31 on the upper side. At the same time, the spring 41 is in the tensioned state, in which the two ends 41a and 41b are pressed together to the maximum extent.

By means of a pressure exerted manually from above, as illustrated by the arrow 47, the manual actuating element is pushed downwards, as a result of which the manual actuating element 34 is displaced through the opening 35 and into the base body (main body) 31, 32, as can be seen from FIG. 4b.

Once the manual actuating element has been displaced in the direction 47 into the base body (main body) 31, 32, a pivoting movement of the needle holding element 36 takes place from left to right, as illustrated by the arrow 48.

A further pivoting movement of the needle holding element during the lancing operation, as illustrated by the arrow 49, which is brought about by the pressure release of the spring 41, leads to a displacement of the needle holding element and thus of the cam 37 within the groove 43 from left to right. During this displacement, the cam 37 travels past the lowest point of the arc-shaped groove 43. At the same time, the cam arranged on the underside of the needle holding element is displaced within the longitudinal groove 44. This leads to an excursion of the needle tip end 39 at the same time as the pivoting movement is carried out. This is illustrated by the arrow 50 and by a comparison of FIGS. 4a, 4b, 4c with regard to the orientation of the needle tip end 39.

FIG. 4c shows the position of the needle and of the needle holding element 36 after use of the lancing device. In this position, a further pivoting of the needle holding element from left to right is brought about by the pressure release of the spring 41 and thus by an exerted pressure force of the second end 41a of the spring 41 from left to right. This is illustrated by the arrow 41.

As a result of such a pivoting process, due to the arc-shaped configuration of the groove 43, the needle holding element 36 is moved upwards again and to the left in the region of the needle tip end 39 in order to carry out a pivoting movement, as illustrated by the arrow 52. In this state, the needle holding element has once again completely disappeared into the base body (main body).

FIGS. 5a and 5b show a perspective view of the lancing device according to a third embodiment of the invention in the assembled and disassembled state. The lancing device according to the third embodiment of the invention has an upper base body (main body) half shell 61 and a lower base body (main body) half shell 62 with an opening 63 and a manual actuating element 64.

The manual actuating element 64 is fixedly connected to the top of a needle holding element 66, the needle holding element 66 in turn comprising on both sides two cams 67 so that the latter can be guided within an arc-shaped groove 73, provided on the base body (main body) halves 61, 62, during the lancing operation.

A needle 68 is once again arranged at the bottom of the needle holding element 66 and has a needle tip end 69 which is designed in the manner of a blade.

Once again, a leg spring 71 is fixedly arranged by a first end 71b relative to the base body (main body) 62 in such a way that it is fixed on webs 76a and on a bolt 76b, which are fixedly arranged on an inner side 72 of the base body (main body) 62, as may take place for example by means of an injection-moulding process.

A further bolt 75 serves as a pivot axle for the spring 71.

The arc-shaped groove 73 has at the left-hand and right-hand side two ends 73a and 73b which both point towards the opening 65. The groove 73 itself is advantageously equipped with two angled ends 73a and 73b so as to carry out an optimised pivoting and displacement movement of the needle holding element 66.

FIGS. 6a-6c show a partially open, perspective view of the pivoting device according to the third embodiment of the invention before, during and after use or the lancing operation. In a starting position, as illustrated in FIG. 6a, the spring 71 is in a maximum tensioned state as a result of the ends 71a and 71b being pushed together. In this state, the needle holding element 66 is arranged with the bolt 67 at the left-hand end 73a of the groove 73. Pressure is then applied to the manual actuating element 64, as illustrated by the arrow 77, whereupon the manual actuating element 64 is pushed into the base body (main body) 61, 62 and then the needle holding element is displaced from left to right, as illustrated by the arrow 78, as a result of a pressure release of the leg spring 71 taking place.

A further pivoting of the needle holding element 76 within the groove 73 from left to right, as illustrated by the arrow 79 in FIG. 6b, leads to an excursion of the needle tip end 69 out of the opening 63, as illustrated by the arrow 80. At the same time, the needle tip end 39 carries out a pivoting movement due to being supported during the displacement process by means of a lateral pivotable arm 74 relative to the edge side of the base body (main body) 62 and optionally 61, and thus forces the lower end of the needle holding element to carry out a pivoting movement, optionally due to a spring effect.

FIG. 6c shows the state of the lancing device according to the third embodiment of the invention after use. In this position, the pivoting movement, as illustrated by the arrow 81, of the needle holding element 66 from left to right has been continued until the cam 67 has reached the right-hand end 73b of the groove 73. The leg spring 71 is then in a pressure-released state. By virtue of the cam 67 travelling along the groove 73 in this way, the needle tip end 69 is retracted back into the housing, i.e. the base body (main body) 61, 62, as illustrated by the arrow 82.

All of the features disclosed in the application documents are claimed as essential to the invention in so far as they are novel individually or in combination with respect to the prior art.

### LIST OF REFERENCES

1 base body (main body)
2 base body (main body)
2a inner wall
2b longitudinal groove
4 manual actuating element
6 needle holding element
7 guide cam
8 needle
9 tip end
10 protective cap
11 leg spring
11a second end
11b first end
12 pivoting element
13 control curve section
13a end
13b end
15 rotation axle
17 latching hook
18 protrusion
19 pressure release
20 movement of the needle
21 pressure release
31 base body (main body)
32 base body (main body)
34 manual actuating element
35 upper side
36 needle holding element
38 needle
39 tip end
41 leg spring
41a second end
41b first end
42 inner wall
43 control curve section
43a end
43b end
44 longitudinal groove
45 rotation axle
48 movement of the needle
49 pressure release
50 movement of the needle
51 pressure release
52 movement of the needle
61 base body (main body)
62 base body (main body)
64 manual actuating element
65 upper side
66 needle holding element
68 needle
69 tip end
71 leg spring
71a second end
71b first end
73 control curve section
73a end
73b end
75 rotation axle
78 movement of the needle
79 pressure release
80 movement of the needle
81 pressure release
82 movement of the needle

1. A lancing device for taking blood for medical examinations, comprising a base body, at least one needle which is arranged therein and which can be extended outwards with a tip end, having a needle holder which at least partially encompasses the needle, and a manual actuator for releasing a movement of the needle together with the needle holder relative to the base body, wherein the needle is spring-loaded, wherein one leg spring is provided, the first end of which is attached to the base body and the second end of which is configured to move the needle holder along a predefined control curve section during a pressure release of the leg spring.

2. The lancing device according to claim 1, wherein the control curve section is arranged as a curved slot in a pivot mounted relative to the base body, and connected to the second end of the leg spring.

3. The lancing device according to claim 2, wherein the needle holder comprises a guide cam configured to engage in the slot of the pivot.

4. The lancing device according to claim 2, wherein the pivot comprises a latching hook for latching into a protrusion of complementary shape arranged on the base body, wherein the latching hook is configured to be released upon movement of the manual actuator.

5. The lancing device according to claim 1, wherein the control curve section comprises a curved groove on an inner wall of the base body and a cam on the needle holder configured to engage in the groove on the inner wall of the base body.

6. The lancing device according to claim 1, wherein the needle holder comprises a pivot member having a first end is connected in a pivotable manner to the needle holder and a second end connected in a pivotable manner to the base body, wherein the pivot member carries out a pivoting movement during a forward and backward displacement movement of the needle holder.

7. The lancing device according to claim 1, wherein the manual actuator is arranged at an upper end of the needle holder located opposite the tip end of the needle and configured to be arrested in the base body.

8. The lancing device according to claim 1, wherein:
an inner wall of the base body comprises a longitudinal groove for receiving a longitudinal guide cam of the needle holder; and
the longitudinal groove extends in a straight line in a tip end region of the needle on the inner wall along the longitudinal axis of the base body.

9. The lancing device according to claim 1, wherein the control curve section is oriented in such a way that its ends are oriented towards an upper side of the base body in a manner pointing away from the tip end of the needle.

10. The lancing device according to claim 1, wherein the leg spring comprises a rotation axle arranged to run perpendicular to the longitudinal axis of the base body in the region of a lower side of the base body in which the tip end of the needle is located.

11. The lancing device according to claim 1, wherein the leg spring comprises a rotation axle arranged to run perpendicular to the longitudinal axis of the base body, which is oriented in the lancing direction, in the region of an upper side of the base body.

12. The lancing device according to claim 1, wherein the control curve section comprises a curved groove on the needle holder and a cam on an inner wall of the base body configured to engage in the groove on the needle holder.

13. A lancing device for taking blood for medical examinations, comprising:
a base body;
at least one needle having a tip end, the needle movably attached within the base body and configured such that the tip end may extend outwards from the base body;
a needle holder which at least partially encompasses the needle;
a manual actuator for releasing a movement of the needle together with the needle holder relative to the base body; and
a leg spring having first and second ends, wherein the first end is attached to the base body and the second end is configured to move the needle holder along a predefined control curve section during a pressure release of the leg spring.

14. The lancing device of claim 13, wherein:
the control curve section comprises a pivot connected to the second end of the leg spring configured to move relative to the base body, the pivot having a curved slot; and
the needle holder comprises a guide cam that engages the curved slot of the pivot.

15. The lancing device of claim 14, wherein:
the base body has a latching protrusion; and
the pivot has a latching hook configured to latch into the latching protrusion of the base body and movably connected to the manual actuator, wherein the latching hook is releasable from the latching protrusion upon movement of the manual actuator.

16. The lancing device of claim 13, wherein:
the control curve section comprises a curved groove on an inner wall of the base body; and
the needle holder comprises a guide cam configured to engage the curved groove on the inner wall of the base body.

17. The lancing device of claim 13, wherein:
the control curve section comprises a curved groove on the needle holder; and
the base body comprises a guide cam configures to engage the curved groove on the needle holder.

18. The lancing device of claim 13, further comprising a pivot having a first end movably connected to the needle holder a second end movably connected to the base body, wherein the pivot is configured to move during a forward and backward displacement movement of the needle holder.

19. The lancing device of claim 13, wherein:
the needle holder has a lower end proximate the tip end of the needle and an upper end opposite the lower end; and
the manual actuator is arranged at the upper end of the needle holder and configured to be arrested in the base body.

20. The lancing device of claim 13, wherein:
the needle holder comprises a longitudinal guide cam; and
the base body comprises a longitudinal groove on an inner wall of the base body configured to receive the longitudinal guide cam, wherein the longitudinal groove extends in a substantially straight line in the tip end region of the needle on the inner wall along the longitudinal axis of the base body.

21. The lancing device of claim 13, wherein the control curve section is configured with a curve having first and second ends oriented substantially pointing away from the tip end of the needle.

22. The lancing device of claim 13, wherein:
the base body comprises upper and lower sides; and
the leg spring comprises a rotation axle configured to run substantially perpendicular to the longitudinal axis of the base body in the region of the lower side of the base body.

23. The lancing device of claim 13, wherein:
the base body comprises upper and lower sides; and
the leg spring comprises a rotation axle configured to run substantially perpendicular to the longitudinal axis of the base body in the region of the upper side of the base body.
